(19) United States
(12) Patent Application Publication
(10) Pub. No.: US 2006/0041194 A1
(43) Pub. Date: Feb. 23, 2006

(54) Surgical gripper with foldable head

(76) Inventors: Mark Sorochkin, Nazareth Illit (IL); Eleonora Sorochkin, Nazareth Illit (IL)

Correspondence Address: DR. MARK FRIEDMAN LTD.;C/o Bill Polkinghom, Discovery Dispatch, 9003 Florin Way, Upper Marlboro, MD, 20772, US

(21) Appl. No.: 10/922,972

(22) Filed: Aug. 23, 2004

(51) Int. Cl. A61B 1/32

(52) U.S. Cl. 600/201

(57) ABSTRACT

The surgical gripper/retractor of the present invention provides a foldable gripping head that may be deployed in a folded position for insertion into a body cavity of a patient. Once so inserted, the displaceable head components of the foldable gripping head may be rotated to an open position so as to expose the tissue contact surfaces. The tissue contact surfaces are configured so as to be displaceable into the corresponding head component. In such a configuration, once the tissue contact surfaces are brought into abutment with the desired tissue, the tissue contact surfaces are displaced into the corresponding head component, thereby creating sufficient suction force to provide temporary adhesion to the tissue.

### FIELD AND BACKGROUND OF THE INVENTION

The present invention relates to surgical retractors and, in particular, it concerns a surgical gripper with a foldable gripping head that utilizes suction force to provide temporary adhesion to the tissue.

It is generally known in laparoscopic surgery to provide a small lumen into which the retractors are inserted. Frequently, it is desirable to provide a surgical retractor that can be introduced into the patient's body through the small incision and thereafter unfolded or opened outwardly inside the inflated body for engaging either small or large tissue masses.

Examples of such surgical retractors include U.S. Pat. No. 5,195,505 to Josefsen, and U.S. Pat. No. 5,607,450 to Zvenyatsky, et al.

Josefsen describes a surgical retractor with a plurality of interleaved retractor blades pivotally mounted at the distal end of a tubular housing and are movable between a closed position, wherein the blades are in stacked relation, and an open position, wherein the blades are deployed in an interleaved fan configuration. Such a device can provide only the ability to push tissue out to the desired location.

Zvenyatsky, et al. discloses a surgical retractor with opposing finger-like elements configured to grip the tissue therebetween. Retractors of this style provide more flexibility with regard to the direction of tissue movement produced by the retractor. However, tissue injury becomes an issue due to the pressure of the opposing finger-like elements.

The example of a vacuum actuated surgical retractor that can be used to retract the body organs is illustrated in U.S. Pat. No. 6,090,041 to Clark, et al. The Clark, et al. retractor, however, is not foldable and therefore cannot provide a retractor having an enlargeable contact surface that can be inserted through a small incision or an orifice into a patient's body. As disclosed, Clark, et al., does not provide a retractor with sufficient contact surface area for making secure enough engagement to retract all sizes of tissue and therefore may not be suitable for some laparoscopic procedures.

U.S. Pat. No. 6,743,220 to Sheffer, et al. discloses a surgical instrument that includes an elongate shaft having two ends with a handle at a first end of the shaft, and a grasping tip at a second end of the shaft. The grasping tip includes a body housing at least one suction element, wherein the at least one suction element mechanically produces suction locally (distally) within the grasping tip. However, Sheffer, et al. clear teaches in column 7, on lines 57-61 that “. . . each of the at least one suction cup acts to mechanically produce suction locally (at the distal site) and . . . is not in fluid connection to a vacuum lumen or connected to an external source of vacuum suction.”

There is therefore a need for a surgical gripper with a foldable gripping head that utilizes suction force to provide temporary adhesion to the tissue. It would be beneficial if the folding head of the gripper allowed for placement the retractor head into a patient body laparoscopically in order to position the gripper within the body in regions of limited accessibility.

### SUMMARY OF THE INVENTION

The present invention is a surgical gripper with a foldable gripping head that utilizes suction force to provide temporary adhesion to the tissue.

According to the teachings of the present invention there is provided, A surgical gripper for gripping body tissue, the surgical gripper comprising:(a) a support rod; and (b) at least a first head component displaceably mounted to a distal end of the support rod , the at least first head component including at least a first tissue contact surface configured to temporarily adhere to the tissue by suction force, the at least first head component configured to alternate between a closed position, in which the at least first tissue contact surface is closed against another surface, for insertion into and out of a patient's body, and an open position for gripping the tissue.

According to a further teaching of the present invention, the at least a first head component is rotatable about an axis that is substantially parallel to a longitudinal axis of the support rod.

According to a further teaching of the present invention, there is also provided, a base head component having at least a second tissue contact surface configured to temporarily adhere to the tissue by suction force, the base head component being fixedly attached to the distal end of the support rod, the base head component associated with the at least a first head component such that in the closed position the first and the second tissue contact surfaces are closed against each other.

According to a further teaching of the present invention, the at least a first head component is hingedly attached to the base head component.

According to a further teaching of the present invention, an outer shape of the at least a first head component and the base head component when in the closed position is substantially cylindrical.

According to a further teaching of the present invention, rotation of the at least a first head component about the axis is remotely actuated by a rotatable adjustment wheel that is mechanically linked to the at least a first head component.

According to a further teaching of the present invention, the at least a first head component is rotatable about an axis that is substantially perpendicular to a longitudinal axis of the support rod.

According to a further teaching of the present invention, there is also provided at least a second displaceable head component having at least a second tissue contact surface configured to temporarily adhere to the tissue by suction force, the at least a second displaceable head component is rotatable about an axis that is substantially perpendicular to a longitudinal axis of the support rod, and the at least a second displaceable head component is associated with the at least a first head component such that in the closed position the first and the second tissue contact surfaces are closed against each other.

According to a further teaching of the present invention, the at least a first head component and the at least a second displaceable head component are hingedly attached to the distal end of the support rod.

According to a further teaching of the present invention, an outer shape of the at least a first head component and the base head component when in the closed position is substantially cylindrical.

According to a further teaching of the present invention, rotation of the at least a first head component and the at least a second displaceable head component about the axis is remotely actuated by a rotatable adjustment wheel that is mechanically linked to the at least a first head component and the at least a second displaceable head component.

According to a further teaching of the present invention, at least a first tissue contact surface is configured such that at least a suction portion of the first tissue contact surface is displaceable into the at least a first head component, and when the tissue contact surface is in contact with the tissue a seal line between the first tissue contact surface and the tissue substantially circumscribes the suction portion, such that when the suction portion is displaced into the head component, a suction force is created between the tissue contact surface and the tissue, thereby providing temporary adhesion to the tissue.

According to a further teaching of the present invention, the displaceable tissue contact surface includes at least one rolling diaphragm fixedly attached along a periphery to the at least a first head component so as to be displaceable into the at least a first head component.

According to a further teaching of the present invention, the at least a first head component includes at least one chamber into which the rolling diaphragm is displaced.

According to a further teaching of the present invention, the at least one chamber and the at least one rolling diaphragm are implemented as a plurality of the chambers and a corresponding plurality of the rolling diaphragms.

According to a further teaching of the present invention, vacuum pressure is used to actuate displacement of the displaceable tissue contact surface.

There is also provided according to the teachings of the present invention, a surgical gripper for gripping body tissue, the surgical gripper comprising: (a) a support rod; (b) at least one head component mounted to a distal end of the support rod; and (c) at least a first tissue contact surface configured such that at least a suction portion of the first tissue contact surface is displaceable into the at least a first head component, and when the tissue contact surface is in contact with the tissue a seal line between the first tissue contact surface and the tissue substantially circumscribes the suction portion, such that when the suction portion is displaced into the head component, a suction force is created between the tissue contact surface and the tissue, thereby providing temporary adhesion to the tissue, the displaceable tissue contact surface being a barrier to prevent the introduction of foreign material into the mechanism of the surgical gripper, displacement of the suction portion being actuated by vacuum pressure.

According to a further teaching of the present invention, the at least a first displaceable tissue contact surface includes at least one rolling diaphragm fixedly attached to the head component.

According to a further teaching of the present invention, the displaceable head component includes at least one chamber into which the rolling diaphragm is displaced.

According to a further teaching of the present invention, the at least one chamber and the at least one rolling diaphragm are implemented as a plurality of the chambers and a corresponding plurality of the rolling diaphragms.

According to a further teaching of the present invention, the at least one head component is implemented as two head components.

According to a further teaching of the present invention, the two head components are configured as: (a) a base head component fixedly attached to the distal end of the support rod; and (b) a displaceable head component displaceably associated with the base head component, the displaceable head component configured to alternate between a closed position, for insertion into and out of a patient body, and an open position for gripping the tissue.

According to a ftrther teaching of the present invention, the displaceable head component is rotatable about an axis that is substantially parallel to a longitudinal axis of the support rod.

According to a further teaching of the present invention, the two head components are configured as two displaceable head components each of which is hingedly attached to the distal end of the support rod.

According to a further teaching of the present invention, the two displaceable head components are rotatable about an axis that is substantially perpendicular to a longitudinal axis of the support rod.

There is also provided according to the teachings of the present invention, a method of gripping body tissue in a patient's body during a surgical procedure, the method comprising: (a) providing a surgical gripper that includes: (i) a support rod; (ii) at least a first head component displaceably mounted to a distal end of the support rod, the at least a first head component configured to alternate between a closed position, in which the at least first tissue contact surface is closed against another surface, for insertion into and out of a patient's body, and an open position for gripping the tissue; and (iii) at least a first tissue contact surface configured such that at least a suction portion of the first tissue contact surface is displaceable into the at least a first head component, and when the tissue contact surface is in contact with the tissue a seal line between the first tissue contact surface and the tissue substantially circumscribes the suction portion, such that when the suction portion is displaced into the head component, a suction force is created between the tissue contact surface and the tissue, thereby providing temporary adhesion to the tissue, the displaceable tissue contact surface being a barrier to prevent the introduction of foreign material into the mechanism of the surgical gripper; (b) inserting the surgical gripper, while in a closed position, into the patient's body; (c) displacing the at least a first head component to an open position; (d) positioning a distal end of the surgical gripper such that the tissue contact surface abuts the body tissue; and (e) displacing the tissue contact surface so as to temporarily adhere to the tissue.

According to a further teaching of the present invention, the temporary adhesion to the tissue is implemented as a suction force created between the tissue contact surface and the tissue.

According to a further teaching of the present invention, the at least a first displaceable tissue contact surface is implemented with at least one rolling diaphragm fixedly attached to the head component.

According to a further teaching of the present invention, the displaceable head component is implemented with at least one chamber into which the rolling diaphragm is displaced.

According to a further teaching of the present invention, the at least one chamber and the at least one rolling diaphragm are implemented as a plurality of the chambers and a corresponding plurality of the rolling diaphragms.

According to a further teaching of the present invention, the displacing the tissue contact surface is accomplished by use of vacuum pressure.

According to a further teaching of the present invention, the at least one head component is implemented as two head components.

According to a further teaching of the present invention, the two head components are implemented as: (a) a base head component fixedly attached to the distal end of the support rod; and (b) a displaceable head component displaceably associated with the base head component, the displaceable head component configured to alternate between a closed position, such that the tissue contact surfaces of the base head component and the displaceable head component are closed against each other, for insertion into and out of a patient body, and an open position for gripping the tissue.

According to a further teaching of the present invention, there is also provided rotating the displaceable head component about an axis that is substantially parallel to a longitudinal axis of.the support rod.

According to a further teaching of the present invention, the two head components are implemented as two displaceable head components each of which is hingedly attached to the distal end of the support rod.

According to a further teaching of the present invention, there is also provided rotating the two displaceable head components about an axis that is substantially perpendicular to a longitudinal axis of the support rod.

### BRIEF DESCRIPTION OF THE DRAWINGS

The invention is herein described, by way of example only, with reference to the accompanying drawings, wherein:

FIG. 1 is a perspective view of a first preferred embodiment of the surgical gripper constructed and operative according to the teachings of the present invention, with the folding head in a closed position;

FIG. 2 is a perspective view of the embodiment of FIG. 1, with the folding head in an open position;

FIG. 3 is an exploded view of the embodiment of FIG. 1;

FIG. 4 is a cross sectional view of the embodiment of FIG. 1 taken along line A-A in FIG. 2;

FIG. 5 is a perspective view of the base head component of the embodiment of FIG. 1;

FIG. 6 is an exploded view of the base head component of FIG. 5;

FIG. 7 is a front view of the base head component of FIG. 5;

FIG. 8 is a cross sectional view of the base head component of FIG. 5 taken along line B-B in FIG. 7;

FIG. 9 is a partly enlarged cross-sectional view of the base head component of FIG. 5.

FIG. 10 is a perspective view of the displaceable head component assembly of the embodiment of FIG. 1;

FIG. 11 is an exploded view of the displaceable head component assembly of the embodiment of FIG. 10;

FIG. 12 is a perspective view of a second preferred embodiment of the surgical gripper constructed and operative according to the teachings of the present invention, with the folding head in a closed position;

FIG. 13 is a perspective view of the embodiment of FIG. 12 with the folding head in an open position;

FIG. 14 is an exploded view of the embodiment of FIG. 12;

FIG. 15 is a cross-sectional view of the embodiment of FIG. 12 taken along line C-C in FIG. 13;

FIG. 16 is a perspective view of the displaceable head component of the embodiment of FIG. 12;

FIG. 17 is an exploded view of the displaceable head component of the embodiment of FIG. 16;

FIG. 18 is a front view of the displaceable head component of the embodiment of FIG. 16; and

FIG. 19 is a cross-sectional view of the displaceable head component of the embodiment of FIG. 16 taken alorig line E-E in FIG. 18.

### DESCRIPTION OF THE PREFERRED EMBODIMENTS

The present invention is a surgical gripper with a foldable gripping head that utilizes suction force to provide temporary adhesion to the tissue.

The principles and operation of a surgical gripper according to the present invention may be better understood with reference to the drawings and the accompanying description.

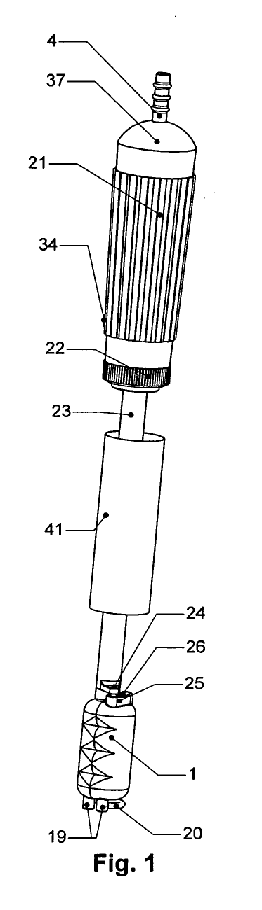

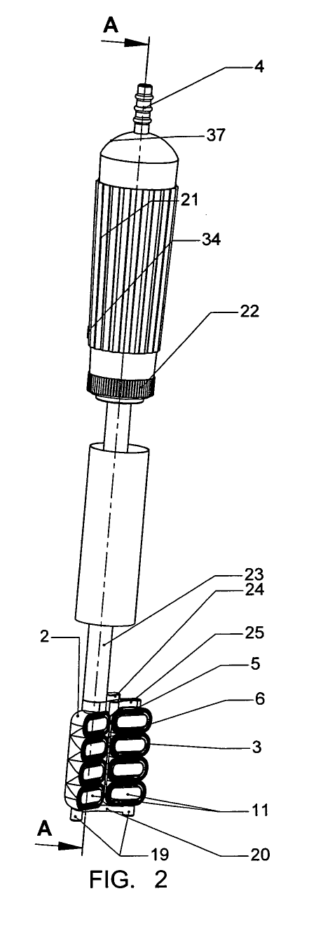

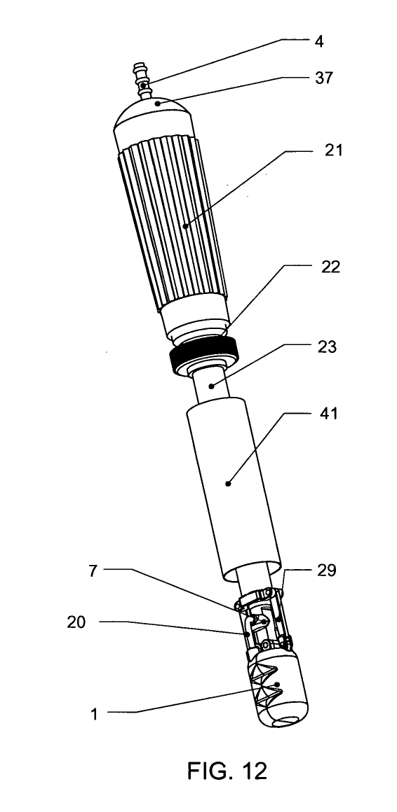

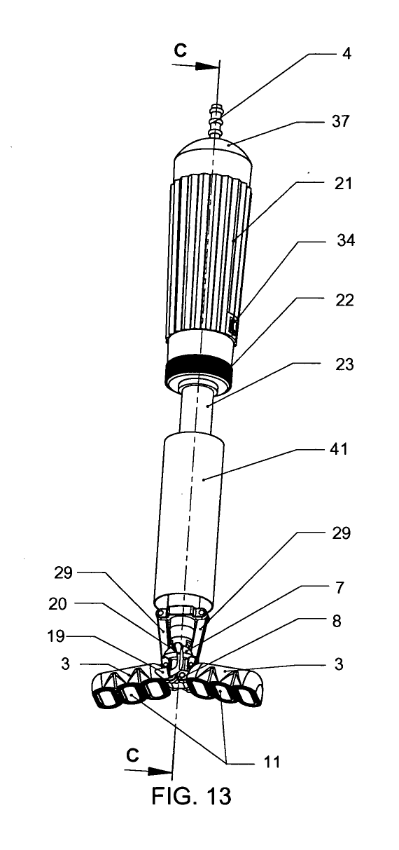

By way of introduction, the surgical gripper/retractor of the present invention is particularly useful in laparoscopic surgery. However, when used during substantially any surgical procedure, it will advance patient safety due to the non-intervention type of engagement between the retractor and the tissue or body organ being retracted. The surgical gripper/retractor of the present invention provides a foldable gripping head 1 that may be deployed in a closed position (FIGS. 1 and 12) for insertion into the body cavity of a patient. Once so inserted, the displaceable head components of the foldable gripping head 1 may be rotated to an open position (FIGS. 2 and 13) so as to expose the tissue contact surfaces. The tissue contact surfaces are configured with displaceable suction portions that are displaceable into the corresponding head component. It should be noted that the tissue contact surfaces also serve to prevent the introduction of body fluids and small unattached body tissues into the mechanism of the present invention, or the vacuum source. That is, the tissue contact surfaces act as a barrier between the two mediums, the patient and the surgical gripper/retractor. In such a configuration, once the tissue contact surfaces are brought into abutment with the desired tissue, the suction portions are displaced into the corresponding head component, thereby creating a suction bond between the tissue contact surfaces and the tissue being gasped. That is to say, displacement of the tissue contact surfaces into the corresponding head component generates sufficient suction force to provide temporary adhesion to the tissue. The preferred embodiments discussed herein relate to displacement of the tissue contact surfaces by the use of vacuum pressure generated inside the head components, thereby drawing the tissue contact surfaces into the head components. However, alterative methods of displacement of the tissue contact surface, such as but not limited to, mechanical, electrical, magnetic, electro-magnetic, and piezoelectric mechanisms are within the scope of the present invention.

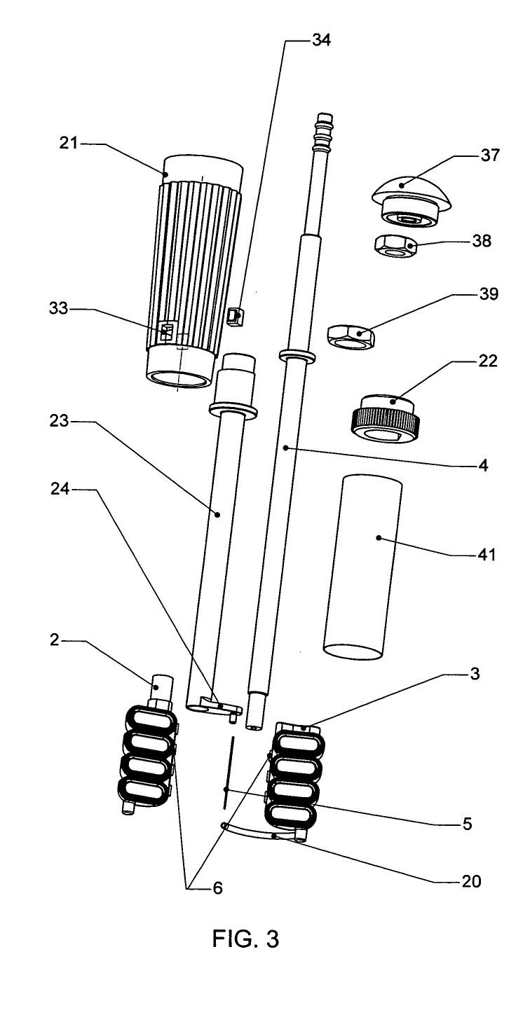

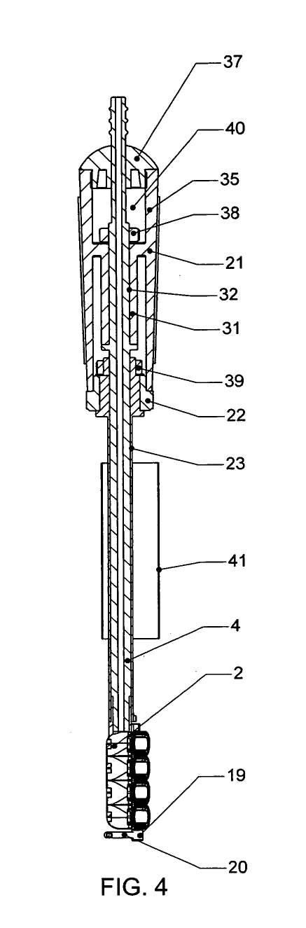

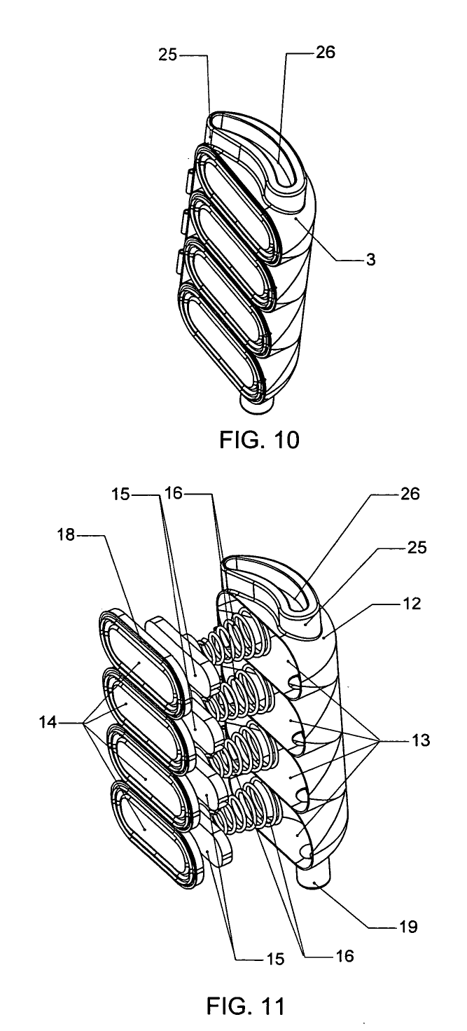

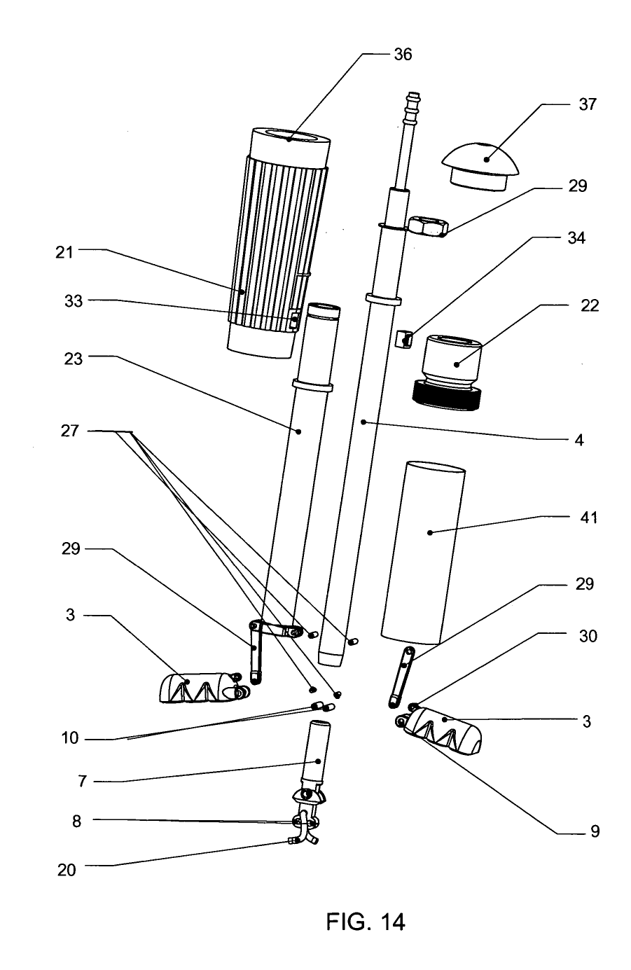

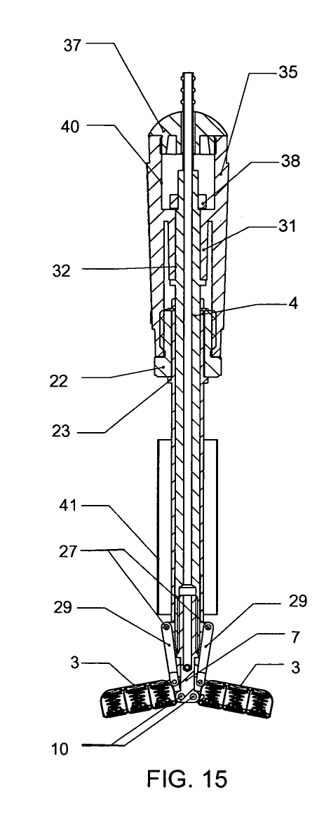

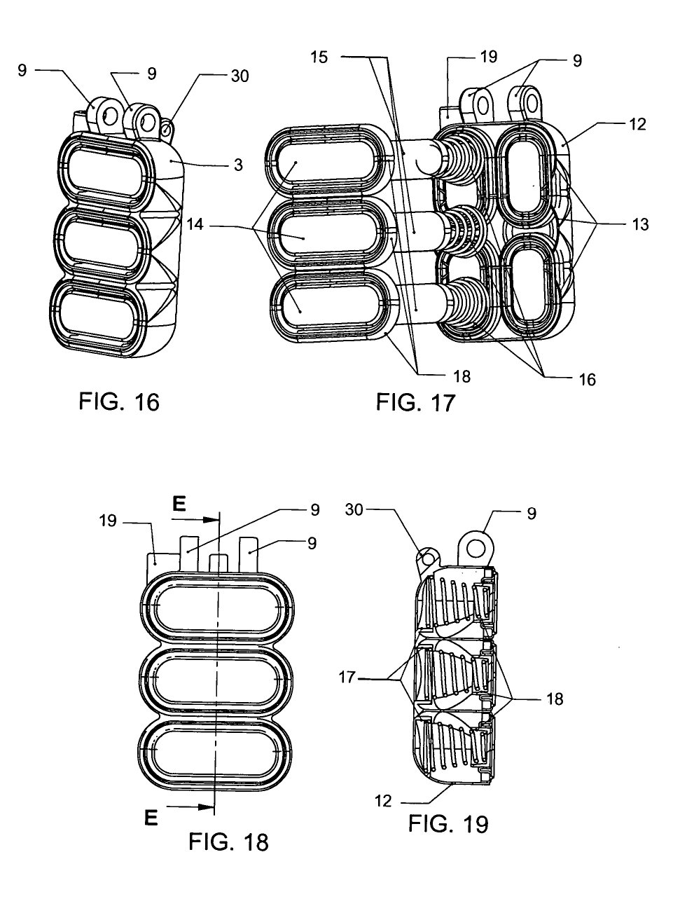

Two preferred embodiments of the present invention are discussed herein. A first preferred embodiment in which the displaceable head component 3 rotates about an axis that is parallel to the longitudinal axis of the surgical gripper/retractor will be discussed with regard to FIGS. 1-11. A second preferred embodiment in which two displaceable head components 3 rotate about an axis that is perpendicular to the longitudinal axis of the surgical gripper/retractor will be discussed with regard to FIGS. 12-19.

It will be understood that the surgical gripper/retractor of the present invention may be used with most trocar instruments and therefore any specific association with a particular trocar assembly is not the concern of this application and therefore, a trocar assembly is schematically represented by item 41 in the Figures.

It should be noted that in the drawings and the following description, components common to both embodiments have been given the same reference number.

Referring now to the drawings and in particular the embodiment of FIGS. 1-11, FIG. 1 illustrates a first preferred embodiment of the present invention with the folding head 1 deployed in a closed position for insertion into the body cavity of a patient. FIG. 2 illustrates the embodiment of FIG. 1 with the folding head deployed in an open position ready for temporary adhesion to tissue.

The folding head of this embodiment includes a base head component 2 and a displaceable head component 3. The base head component 2 is fixedly mounted at the distal end of the support rod 4 that constitutes the main support of the surgical gripper/retractor. Here, the displaceable head component 3 is pivotally mounted with respect base head component 2 so as to rotate about pin 5 located within hinge 6, which is integrally formed with base head component 2. It will be appreciated that the base head component 2 and the displaceable head component 3 may be integrally formed with an integrally formed hinge interconnecting them.

The base head component 2 and displaceable head component 3 have, on the whole, a similar construction and preferably have a substantially semicircular cross-sectional contour so as to form a cylinder when deployed in the closed position with the two contact surfaces 11 are closed against each other. Such a form provides a suitable shape for insertion of the gripper/retractor into patient's body through the trocar 41.

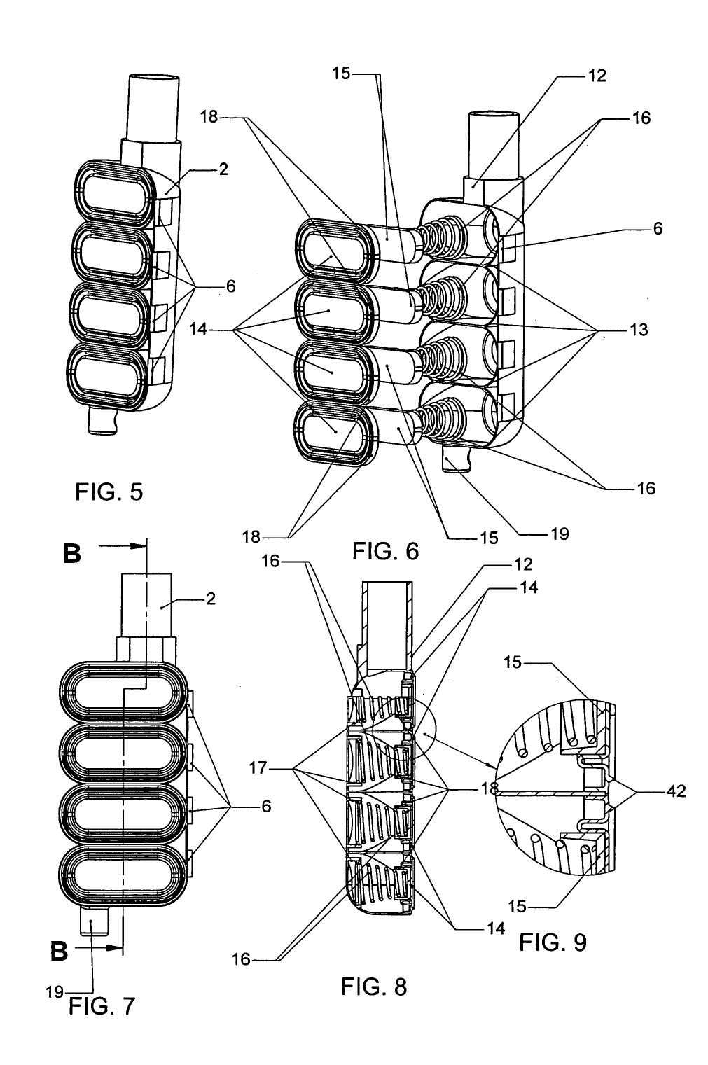

Turning now to the configuration of the head components 2 and 3 as illustrated in FIGS. 5-9, each of the head components 2 and 3 include a housing 12 composed of a plurality of flattened oval shaped chambers 13 each configured to receive a rolling diaphragm 14, a flexible piston 15 abutting the rolling diaphragms 14, and biasing element preferably, but not limited to, a tapered coil spring 16. The tapered coil spring 16 biases the flexible piston 15 so as to keep the rolling diaphragm 14 in an outward displacement until a vacuum state is created inside the head component thereby drawing the displaceable suction portion of the rolling diaphragms 14 into the head component. The outer edges of the rolling diaphragms 14 are fixedly secured to the sidewall of the chambers 13, preferably by, but non limited to, an adhesive. It will be appreciated that the size, shape and number of the chambers 13 should be considered a design choice and the descriptions of these features herein are intended as non-limiting examples only.

As seen in FIG. 8 each of the chambers 13 are provided with an open hollow projection 17 for receiving therein one end of the tapered coil spring 16, the other end of which abuts the flexible piston 15, which is also provided with a hollow projection 18 to receive therein the corresponding end of the coil spring 16. The flexible piston 15 is preferably secured to the rolling diaphragm 14 with adhesive. However, any suitable attachment method is within the scope of the present invention. It should be appreciated that the rolling diaphragm 14 forms a flexible seal that is configured to permit piston stroke while completely eliminating sliding friction between the piston 15 and any associated cylinder. Therefore, there no need for finely machined surfaces and the tolerances required were the piston to be encased in an associated cylinder.

The outer flange 18 of the rolling diaphragm 14 is fastened to the wall of chamber 13, thereby preventing blow-by leakage and vacuum loss. It will be readily appreciated that the employment of such a diaphragm also serves to prevent the aspiration of body fluids and small unattached body tissues into the mechanism of the present invention, or the vacuum source. The diaphragm may be configured from, for example, woven fabric with a thin layer of elastomer having a total thickness 0.2-0.4 mm. However, any suitable material with the required elastomeric properties, such as but not limited to, rubber, plastics, and latex, may be used.

The tissue contact surface 11 of the rolling diaphragm 14 is provided with a triangular ridge 42 (see FIG. 9). When the tissue contact surface 11 is brought into contact with a tissue mass or body organ, contact of the triangular ridge 42 against the tissue forms a seal line that substantially circumscribes the suction portion of the rolling diaphragm 14. It should be noted that the rolling diaphragm 14 may be configure as a plurality of individual diaphragms, one for each chamber 13, as illustrated herein, or as a single diaphragm configured to cover all of the chambers 13 in one head component. Such a single diaphragm would, by necessity, include separate suction portions corresponding to, and operative with, each of the chambers 13 in the corresponding head component.

In this first preferred embodiment of the present invention, housing 12 of the base head component 2 is in fluid communication with the hollow support rod 4, which is connected to a vacuum source. Each of the housings 12 of the head components 2 and 3 are configured with integrally formed nipples 19 so as to receive flexible hose 20 deployed therebetween to provide fluid communication between the displaceable head component 3 and the base head component 2. Thereby establishing fluid communication between the vacuum source and both the displaceable head component 3 and the base head component 2. Therefore, the creation of a vacuum state inside the head components 2 and 3 results in displacement of the suction portions rolling diaphragms 14 into the corresponding chambers 13 of the head components 2 and 3.

Turning now to the mechanism for actuating rotation of the displaceable head component 3, this embodiment of the surgical gripper/retractor is provided with an actuating mechanism that includes an adjustment wheel 22, which is associated with the handle 21, and a tubular drive rod 23. The handle 21 is attached to the proximal end of the portion of the hollow support rod 4 extending throughout the handle 21 by nut 38.

The tubular drive rod 23 is rotatably mounted around the support rod 4. At the distal end of the tubular drive rod 23 is an actuating arm 24 that extends perpendicularly from the tubular drive rod 23. The actuating arm 24 includes an actuating pin 24a configured to engage slot 26 configured in projection 25 of the housing 12 of the displaceable head component 3. The actuating arm 24 may be integrally formed with tubular drive rod 23, or it may be a separate element affixed to the tubular drive rod 23.

The adjustment wheel 22 is fixedly attached to the proximal end of the tubular drive rod 23 with nut 39 and abuts the handle 21. For convenience the outer surface of the adjustment wheel 22 is knurled to avoid slippage of the surgeon's fingers during use. Configured in this way, by rotating the adjustment wheel 22 the position of the displaceable head component 3 may be varied and the displaceable head component 3 may be suitably spaced away from the base head component 2 as required.

In a preferred construction, handle 21 be formed as a unitarily molded member, although an integral assembly of the various parts could also be implemented. Further, in the preferred embodiments, the handle 21 is provided with an axial projection 31 therein that contain a centered passage 32 extending longitudinally therethrough that receives support rod 4.

Also associated with the handle 21 is switch 34, deployed in opening 33. At the proximal end of the handle 21 body 35 forms rearward opening passage 36 adapted to receive the threaded plug 37 provided with a hole, which is in axial alignment with centered passage 32 of the projection 31. The body 35 and plug 37 collectively define an inner space 40, which receives wires connecting the switch 34 to a vacuum control unit valve (not shown). Alternatively, switch 34 may be mechanically linked to a valve located in the handle 21. Switch 34 may alternatively be configured to activate a device operative according to any of the alternate displacement methods mentioned above.

Turning now to a second preferred embodiment of the present invention, the two head components 3 illustrated in FIGS. 12-19 rotate about an axis that is perpendicular to the longitudinal axis of the surgical gripper/retractor. That is, both head components 3 are displaceable with respect to the support rod 4. It is understood that the structure and function of the contact surfaces, including the rolling diaphragm 14, flexible piston 15, and tapered coil spring 16, and their association within, and to, the displaceable head components 3, is substantially the same as discussed above with regard to the first preferred embodiment and therefore will not be discussed now with regard to the second preferred embodiment.

Here, in the second preferred embodiment of the present invention, both head components 3 are configured as displaceable head components 3 and are hingedly attached to connector 7 which is provided with rounded projections 8 integrally formed with connector 7. The projections 8 are configured so as to receive arms 9 of displaceable head components 3. Arms 9 and projections 8 have throughbores for receiving pin 10 to achieve moveable attachment between the displaceable head components 3 and the connector 7. The adjustment wheel 22 is threadably attached to the distal end of the handle 21 and rotatably interconnected to the proximal end of the tubular drive rod 23. The tubular drive rod 23 is slidebly mounted on the support rod 4.

The tubular drive rod 23 is provided on the distal end with projections 28, which are configured to receive pins 27 that pass through thoroughbores in the distal end of the connecting rod 29. The proximal end of the connecting rod 29 is hingedly attached to the projection 30 integrally formed with housing 12 of the displaceable head component 3. Therefore, when the adjustment wheel 22 is rotated it is displaced into or out of the handle 21, thereby causing linear movement of the tubular drive rod 23 and rotating the displaceable head components 3 between a closed position and an open (fully extended) position.

In this embodiment flexible hoses 20 extend from the connector 7 to the housings 12 of the each of the displaceable head component 3 so as to provide fluid communication therebetween, thereby providing the vacuum pressure to actuate the tissue contact surfaces (rolling diaphragms), as was discussed above. Therefore, switch 34 may be configured as is described above.

The following steps may be included in a method for the use of any embodiments of the present invention herein described:
1—Insertion of the surgical gripper/retractor, while in a closed position, into the patient's body;
2—Displacement of the displaceable head component to an open position;
3—Positioning the distal end of the surgical gripper such that the tissue contact surfaces abut the desired body tissue;
4—Displacing the tissue contact surface so as to temporarily adhere to the tissue. That is, creating a vacuum state inside the head components, thereby causing the rolling diaphragms to be displaced into the corresponding chambers in the head components.

It will be readily apparent to one of ordinary skill in the art that the components of the present invention may be constructed from various suitable materials such as, but not limited to, metals, plastics (injection or vacuum molded), polymers, or any combination thereof.

It will be appreciated that the above descriptions are intended only to serve as examples and that many other embodiments are possible within the spirit and the scope of the present invention.

What is claimed is:

1. A surgical gripper for gripping body tissue, the surgical gripper comprising:
(a) a support rod; and
(b) at least a first head component displaceably mounted to a distal end of said support rod , said at least first head component including at least a first tissue contact surface configured to temporarily adhere to the tissue by suction force, said at least first head component configured to alternate between a closed position, in which said at least first tissue contact surface is closed against another surface, for insertion into and out of a patient's body, and an open position for gripping the tissue.

2. The surgical gripper of claim 1, wherein said at least a first head component is rotatable about an axis that is substantially parallel to a longitudinal axis of said support rod.

3. The surgical gripper of claim 2, further including a base head component having at least a second tissue contact surface configured to temporarily adhere to the tissue by suction force, said base head component being fixedly attached to said distal end of said support rod, said base head component associated with said at least a first head component such that in said closed position said first and said second tissue contact surfaces are closed against each other.

4. The surgical gripper of claim 3, wherein said at least a first head component is hingedly attached to said base head component.

5. The surgical gripper of claim 4, wherein an outer shape of said at least a first head component and said base head component when in said closed position is substantially cylindrical.

6. The surgical gripper of claim 4, wherein rotation of said at least a first head component about said axis is remotely actuated by a rotatable adjustment wheel that is mechanically linked to said at least a first head component.

7. The surgical gripper of claim 1, wherein said at least a first head component is rotatable about an axis that is substantially perpendicular to a longitudinal axis of said support rod.

8. The surgical gripper of claim 7, further including at least a second displaceable head component having at least a second tissue contact surface configured to temporarily adhere to the tissue by suction force, said at least a second displaceable head component is rotatable about an axis that is substantially perpendicular to a longitudinal axis of said support rod, and said at least a second displaceable head component is associated with said at least a first head component such that in said closed position said first and said second tissue contact surfaces are closed against each other.

9. The surgical gripper of claim 8, wherein said at least a first head component and said at least a second displaceable head component are hingedly attached to said distal end of said support rod.

10. The surgical gripper of claim 9, wherein an outer shape of said at least a first head component and said base head component when in said closed position is substantially cylindrical.

11. The surgical gripper of claim 10, wherein rotation of said at least a first head component and said at least a second displaceable head component about said axis is remotely actuated by a rotatable adjustment wheel that is mechanically linked to said at least a first head component and said at least a second displaceable head component.

12. The surgical gripper of claim 1, wherein at least a first tissue contact surface is configured such that at least a suction portion of said first tissue contact surface is displaceable into said at least a first head component, and when said tissue contact surface is in contact with the tissue a seal line between said first tissue contact surface and the tissue substantially circumscribes said suction portion, such that when said suction portion is displaced into said head component, a suction force is created between the tissue contact surface and the tissue, thereby providing temporary adhesion to the tissue.

13. The surgical gripper of claim 12, wherein said displaceable tissue contact surface includes at least one rolling diaphragm fixedly attached along a periphery to said at least a first head component so as to be displaceable into said at least a first head component.

14. The surgical gripper of claim 13, wherein said at least a first head component includes at least one chamber into which said rolling diaphragm is displaced.

15. The surgical gripper of claim 14, wherein said at least one chamber and said at least one rolling diaphragm are implemented as a plurality of said chambers and a corresponding plurality of said rolling diaphragms.

16. The surgical gripper of claim 12, wherein vacuum pressure is used to actuate displacement of said displaceable tissue contact surface.

17. A surgical gripper for gripping body tissue, the surgical gripper comprising:
(a) a support rod;
(b) at least one head component mounted to a distal end of said support rod; and
(c) at least a first tissue contact surface configured such that at least a suction portion of said first tissue contact surface is displaceable into said at least a first head component, and when said tissue contact surface is in contact with the tissue a seal line between said first tissue contact surface and the tissue substantially circumscribes said suction portion, such that when said suction portion is displaced into said head component, a suction force is created between the tissue contact surface and the tissue, thereby providing temporary adhesion to the tissue, said displaceable tissue contact surface being a barrier to prevent the introduction of foreign material into the mechanism of the surgical gripper, displacement of said suction portion being actuated by vacuum pressure.

18. The surgical gripper of claim 17, wherein said at least a first displaceable tissue contact surface includes at least one rolling diaphragm fixedly attached to said head component.

19. The surgical gripper of claim 18, wherein said displaceable head component includes at least one chamber into which said rolling diaphragm is displaced.

20. The surgical gripper of claim 19, wherein said at least one chamber and said at least one rolling diaphragm are implemented as a plurality of said chambers and a corresponding plurality of said rolling diaphragms.

21. The surgical gripper of claim 17, wherein said at least one head component is implemented as two head components.

22. The surgical gripper of claim 21, wherein said two head components are configured as:
(a) a base head component fixedly attached to said distal end of said support rod; and
(b) a displaceable head component displaceably associated with said base head component, said displaceable head component configured to alternate between a closed position, for insertion into and out of a patient body, and an open position for gripping the tissue.

23. The surgical gripper of claim 22, wherein said displaceable head component is rotatable about an axis that is substantially parallel to a longitudinal axis of said support rod.

24. The surgical gripper of claim 21, wherein said two head components are configured as two displaceable head components each of which is hingedly attached to said distal end of said support rod.

25. The surgical gripper of claim 24, wherein said two displaceable head components are rotatable about an axis that is substantially perpendicular to a longitudinal axis of said support rod.

26. A method of gripping body tissue in a patient's body during a surgical procedure, the method comprising:
(a) providing a surgical gripper that includes:
(i) a support rod;
(ii) at least a first head component displaceably mounted to a distal end of said support rod, said at least a first head component configured to alternate between a closed position, in which said at least first tissue contact surface is closed against another surface, for insertion into and out of a patient's body, and an open position for gripping the tissue; and
(iii) at least a first tissue contact surface configured such that at least a suction portion of said first tissue contact surface is displaceable into said at least a first head component, and when said tissue contact surface is in contact with the tissue a seal line between said first tissue contact surface and the tissue substantially circumscribes said suction portion, such that when said suction portion is displaced into said head component, a suction force is created between the tissue contact surface and the tissue, thereby providing temporary adhesion to the tissue, said displaceable tissue contact surface being a barrier to prevent the introduction of foreign material into the mechanism of the surgical gripper;
(b) inserting said surgical gripper, while in a closed position, into the patient's body;
(c) displacing said at least a first head component to an open position;
(d) positioning a distal end of said surgical gripper such that said tissue contact surface abuts the body tissue; and
(e) displacing said tissue contact surface so as to temporarily adhere to the tissue.

27. The method of claim 26, wherein said temporary adhesion to the tissue is implemented as a suction force created between the tissue contact surface and the tissue.

28. The method of claim 26, wherein said at least a first displaceable tissue contact surface is implemented with at least one rolling diaphragm fixedly attached to said head component.

29. The method of claim 28, wherein said displaceable head component is implemented with at least one chamber into which said rolling diaphragm is displaced.

30. The method of claim 29, wherein said at least one chamber and said at least one rolling diaphragm are implemented as a plurality of said chambers and a corresponding plurality of said rolling diaphragms.

31. The method of claim 26, wherein said displacing said tissue contact surface is accomplished by use of vacuum pressure.

32. The method of claim 26, wherein said at least one head component is implemented as two head components.

33. The method of claim 32, wherein said two head components are implemented as:
(a) a base head component fixedly attached to said distal end of said support rod; and
(b) a displaceable head component displaceably associated with said base head component, said displaceable head component configured to alternate between a closed position, such that said tissue contact surfaces of said base head component and said displaceable head component are closed against each other, for insertion into and out of a patient body, and an open position for gripping the tissue.

34. The method of claim 33, further including rotating said displaceable head component about an axis that is substantially parallel to a longitudinal axis of said support rod.

35. The method of claim 32, wherein said two head components are implemented as two displaceable head components each of which is hingedly attached to said distal end of said support rod.

36. The method of claim 35, further including rotating said two displaceable head components about an axis that is substantially perpendicular to a longitudinal axis of said support rod.
